(19) United States
(12) Patent Application Publication
(10) Pub. No.: US 2026/0140024 A1
(43) Pub. Date: May 21, 2026

(54) WATER-QUALITY DETECTION APPARATUS AND WATER-QUALITY DETECTION METHOD USING THE SAME

(71) Applicant: INDUSTRIAL TECHNOLOGY RESEARCH INSTITUTE, Hsinchu (TW)

(72) Inventors: Tsung-Yu TSAI, Tainan City (TW); Cheng-Da KO, Tainan City (TW); Hung-Sheng LIN, Tainan City (TW); Hsin-Erh WANG, Tainan City (TW)

(73) Assignee: INDUSTRIAL TECHNOLOGY RESEARCH INSTITUTE, Hsinchu (TW)

(21) Appl. No.: 19/001,899

(22) Filed: Dec. 26, 2024

(30) Foreign Application Priority Data
Nov. 20, 2024 (TW) 113144627

(51) Int. Cl. G01N 1/38; G01N 21/77; G01N 21/78; G01N 21/85; G01N 33/18

(52) U.S. Cl. CPC G01N 1/38; G01N 21/78; G01N 21/85; G01N 33/1893; G01N 2021/7783

(57) ABSTRACT

A water-quality detection apparatus includes a detection chamber device, a pump module, a light source, a light sensor and a controller. The pump module is configured to transport a to-be-tested liquid and a reagent to a detection chamber of the detection chamber device. The light source emits detection light to the detection chamber. The light sensor senses the detection light traveling through the detection chamber and generates a detection voltage. The controller performs the following procedures: a voltage sensing procedure, a dilution determination procedure, a dilution procedure and a concentration acquisition procedure. The dilution determination procedure may calculate a voltage ratio between the detection voltage value and a basic-voltage value. When the voltage ratio is greater than a critical value, the concentration acquisition procedure is performed. When the voltage ratio is not greater than the critical value, the dilution procedure is performed.

This application claims the benefit of Taiwan application Serial No. 113144627, filed Nov. 20, 2024, the disclosure of which is incorporated by reference herein in its entirety.

### TECHNICAL FIELD

The disclosure relates in general to a water-quality detection apparatus and a water-quality detection method using the same.

### BACKGROUND

A water-quality detection apparatus may detect a concentration of impurities in a to-be-tested liquid. However, the concentration of the to-be-tested liquid varies widely. The to-be-tested liquid having different concentration ranges requires different water-quality detection apparatus for testing, which increases equipment costs and requires replacement of the water-quality detection apparatus during the testing process, and thus reducing testing efficiency. Therefore, how to improve the aforementioned conventional problems is one of the goals of those in this technical field.

### SUMMARY

According to an embodiment, a water-quality detecting equipment is provided. The water-quality detecting equipment includes a detection chamber device, a pump module, a light source, a light sensor and a controller. The detection chamber device has a detection chamber. The pump module is configured to transport a to-be-tested liquid and a reagent to the detection chamber. The light source is configured to emit a detection light to the detection chamber. The light sensor is configured to sense the detection light traveling through the detection chamber and generate a detection voltage. The controller is electrically connected to the light sensor and the pump module, and configured to perform following procedures: a voltage sensing procedure comprising: reading a detection voltage value of the detection voltage from the light sensor; a dilution determination procedure comprising: calculating a voltage ratio of the detection voltage value and a basic-voltage value, comparing the voltage ratio with a critical value, performing a concentration acquisition procedure when the voltage ratio is greater than the critical value, and performing a to-be-tested liquid dilution procedure when the voltage ratio is not greater than the critical value. Wherein in the concentration acquisition procedure, a detection concentration value of the to-be-tested liquid is obtained according to a dilution rate and the detection voltage value, wherein In the to-be-tested liquid dilution procedure, the dilution rate is updated according to the voltage ratio, and the pump module is driven to transport a diluent by the controller, so that a ratio of a volume sum of the to-be-tested liquid volume of the to-be-tested liquid in the detection chamber and the diluent volume of the diluent to the to-be-tested liquid volume of the to-be-tested liquid is equal to the dilution rate, and the voltage sensing procedure and the dilution determination procedure are performed.

According to an embodiment, a water-quality detecting method is provided. The water-quality detecting method includes the following steps: transporting a to-be-tested liquid and a reagent to a detection chamber of a detection chamber device by a pump module; emitting a detection light to the detection chamber by a light source; sensing the detection light traveling through the detection chamber and generating a detection voltage by a light sensor; performing a voltage sensing procedure by a controller, comprising: reading a detection voltage value of the detection voltage; and performing a dilution determination procedure by the controller, comprising: calculating the voltage ratio of the detection voltage value to a basic-voltage value; comparing the voltage ratio with a critical value; performing a concentration acquisition procedure when the voltage ratio is greater than the critical value; and performing a to-be-tested liquid dilution procedure when the voltage ratio is not greater than the critical value. The concentration acquisition procedures comprises: obtaining a detection concentration value of the to-be-tested liquid according to a dilution rate and the detection voltage value by the controller. The to-be-tested liquid dilution procedure comprises: updating the dilution rate according to the voltage ratio by the controller; and driving the pump module to transport a diluent by the controller, so that a ratio of a volume sum of a to-be-tested liquid volume of the to-be-tested liquid in the detection chamber and the diluent volume of the diluent to the to-be-tested liquid volume of the to-be-tested liquid is equal to the dilution rate, and performing the voltage sensing procedure and the dilution determination procedure.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1A illustrates a functional block diagram of the water-quality detection apparatus according to an embodiment of the disclosure;

FIG. 1B illustrates a schematic diagram of the water-quality detection apparatus in FIG. 1A;

FIG. 1C illustrates a flow chart of a water-quality detection method of the water-quality detection apparatus in FIG. 1B;

FIG. 2A illustrates a functional block diagram of the water-quality detection apparatus according to an embodiment of the disclosure;

FIG. 2B illustrates a schematic diagram of the water-quality detection apparatus in FIG. 2A;

FIG. 2C illustrates a schematic diagram of a detection chamber device in FIG. 2B and a detection base matched with the detection chamber device;

FIG. 2D illustrates a flow chart of a water-quality detection method performed by the water-quality detection apparatus in FIG. 2B;

FIG. 3A illustrates a flow chart of the basic-voltage acquisition procedure in FIG. 2D;

FIG. 3B illustrates a schematic diagram of the detection chamber of the detection chamber device covered with a light-shielding component;

FIG. 4 illustrates a flow chart of the initialization procedure in FIG. 2;

FIG. 5 illustrates a relationship diagram between a color pigmentation time of the to-be-tested liquid and the detection voltage value according to the embodiment of the disclosure;

FIG. 6 illustrates a flow chart of the voltage sensing procedure in FIG. 2D;

FIG. 7 illustrates a flow chart of the dilution determination procedure, the concentration acquisition procedure and the to-be-tested liquid dilution procedure in FIG. 2D;

FIG. 8A illustrates a flow chart of the concentration acquisition procedure in FIG. 2D;

FIG. 8B illustrates a relationship diagram between a standard concentration value and an absorbance according to an embodiment of the in disclosure;

FIG. 9 illustrates a flow chart of the to-be-tested liquid dilution procedure in FIG. 2D according to another embodiment;

FIG. 10 illustrates a schematic diagram of the water-quality detection method of the water-quality detection apparatus in FIG. 2B according to another embodiment;

FIG. 11 illustrates a flow chart of the concentration acquisition procedure in FIG. 10;

FIG. 12 illustrates a flow chart of the to-be-tested liquid dilution procedure in FIG. 10;

FIG. 13A illustrates a functional block diagram of a water-quality detection apparatus according to another embodiment of the disclosure; and

FIG. 13B illustrates a schematic diagram of the water-quality detection apparatus in FIG. 13A.

### DETAILED DESCRIPTION

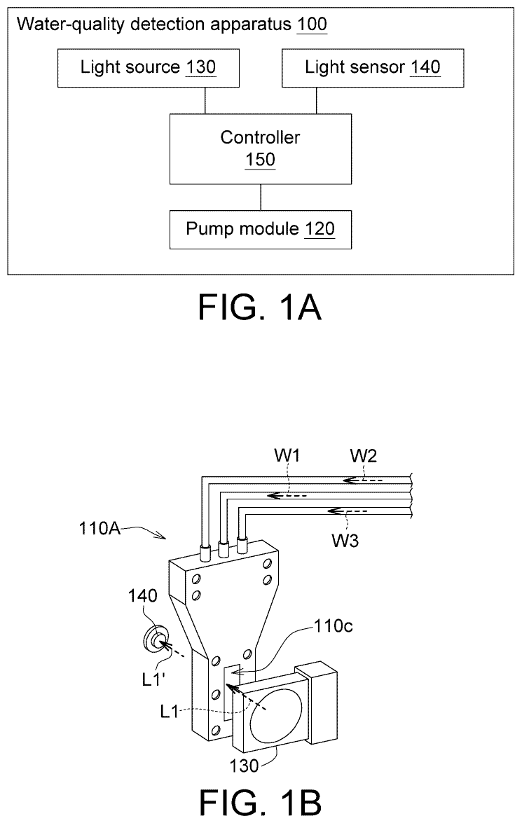

Referring to FIGS. 1A, 1B, and 1C, FIG. 1A illustrates a functional block diagram of the water-quality detection apparatus 100 according to an embodiment of the disclosure, FIG. 1B illustrates a schematic diagram of the water-quality detection apparatus 100 in FIG. 1A, and FIG. 1C illustrates a flow chart of a water-quality detection method of the water-quality detection apparatus in FIG. 1B.

As illustrated in FIGS. 1A and 1B, the water-quality detection apparatus 100 includes a detection chamber device 110A, a pump module 120, a light source 130, a light sensor 140 and a controller 150.

As illustrated in FIGS. 1A to 1C, the detection chamber device 110A has a detection chamber 110c. The pump module 120 is configured to transport the to-be-tested liquid W1 and a first reagent W2 to the detection chamber 110c. The light source 130 is configured to emit a detection light L1. The light sensor 140 is configured to sense the detection light L1′ traveling through the detection chamber 110c and generate a detection voltage. The controller 150 is electrically connected to the light sensor 140 and the pump module 120 and is configured to perform: a voltage sensing procedure S130 to read a detection voltage value VS of the detection voltage of the light sensor; a dilution determination procedure S140 to calculate a voltage ratio VR of the detection voltage value VS to a basic-voltage value Vo, and compare the voltage ratio VR with a critical value; perform a concentration acquisition procedure S150 when the voltage ratio VR is greater than the critical value; perform a to-be-tested liquid dilution procedure S160 when the voltage ratio VR is not greater than the critical value. In the to-be-tested liquid dilution procedure S160, a dilution rate is updated according to the voltage ratio VR, and the pump module 120 is driven to transport a diluent W3, so that a ratio of a volume sum of a to-be-tested liquid volume of the to-be-tested liquid W1 in the detection chamber 110c and a diluent volume of the diluent W3 to the to-be-tested liquid volume of the to-be-tested liquid W1 is equal to the dilution rate, and then the voltage sensing procedure S130 and the dilution determination procedure S140 are performed sequentially again. In the concentration acquisition procedure S150, a detection concentration value S1 of the to-be-tested liquid W1 is obtained according to the dilution rate and the detection voltage value. As a result, the concentration of the to-be-tested liquid W1 (for example, ammonia nitrogen concentration, nitrate nitrogen concentration) may be detected through optical means, and it is ensured that the liquid concentration in the detection chamber 110c is within the range that the light sensor 140 may accurately measure, and an applicable detection concentration range of the water-quality detection apparatus 100 may be effectively expanded.

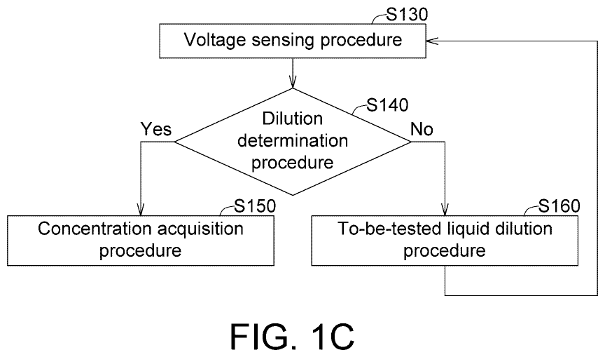

Referring to FIGS. 2A, 2B, 2C and 2D, FIG. 2A illustrates a functional block diagram of the water-quality detection apparatus 200 according to an embodiment of the disclosure, FIG. 2B illustrates a schematic diagram of the water-quality detection apparatus 200 in FIG. 2A, FIG. 2C illustrates a schematic diagram of a detection chamber device 210A in FIG. 2B and a detection base 210B matched with the detection chamber device 210A, and FIG. 2D illustrates a flow chart of a water-quality detection method performed by the water-quality detection apparatus 200 in FIG. 2B.

As illustrated in FIGS. 2A to 2C, the water-quality detection apparatus 200 includes the detection chamber device 210A, a pump module 220, a light source 230, a light sensor 240, a controller 250, a motor 260 and an evacuation valve 270, a to-be-tested liquid container 11 for storing the to-be-tested liquid W1, a first reagent container 12 for storing the first reagent W2 and a diluent container 13 for storing the diluent W3.

As illustrated in FIG. 2A, the controller 250 is electrically connected to the pump module 220, the light source 230, the light sensor 240, the motor 260 and the evacuation valve 270 to control the operation of these components and/or receive the signals from these components. The light source 230 and the light sensor 240 may be disposed in the detection base 210B.

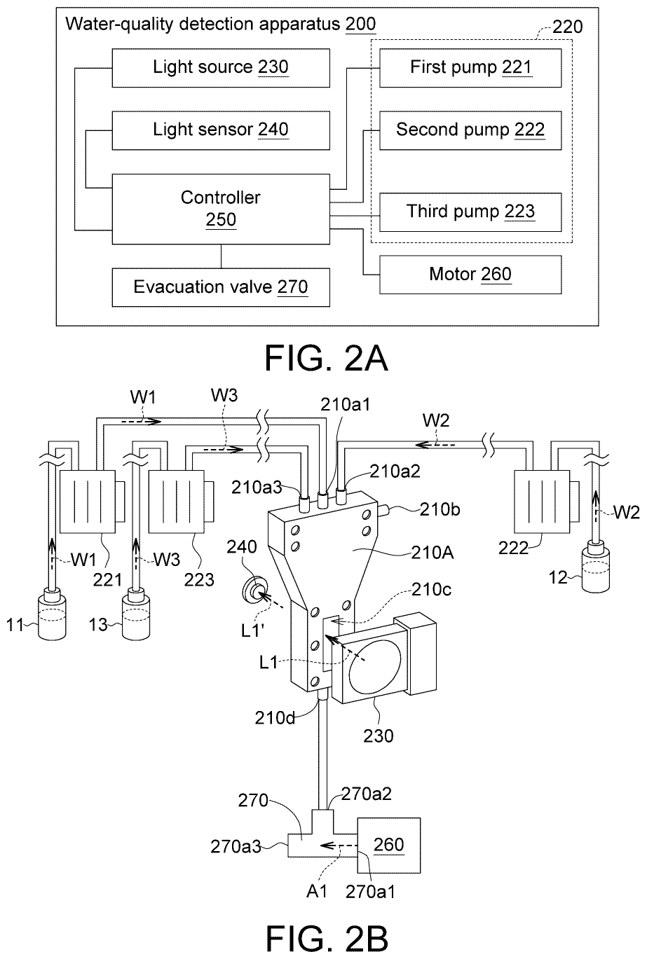

As illustrated in FIGS. 2B to 2C, the detection chamber device 210A may be partially inserted into a recess of the detection base 210B. For example, the detection window may be located in the recess of the detection base 210B to avoid the detection being interfered by ambient light. The detection chamber device 210A has a detection chamber 210c, a first detection window 211 and a second detection window 212 (the second detection window 212 is illustrated in FIG. 3). The first detection window 211 and the second detection window 212 are, for example, light-transmitting windows, so the internal condition of the detection chamber 210c may be seen through the detection windows. The light source 230 may be located at side of the first detection window 211 to emit the detection light L1 toward the first detection window 211. The light sensor 240 may be located at side of the second detection window 212 to receive the detection light L1′ traveling through the detection chamber 210c and generate the detection voltage. Furthermore, the light source 230 and the light sensor 240 are respectively located at opposite sides of the detection chamber 210c. As a result, the detection light L1 emitted by the light source 230 may be received by the light sensor 240 after traveling through the detection chamber 210c. The light source 230 is, for example, an LED light source. In an embodiment, the light source herein is, for example, an ultraviolet light source which may emit the detection light L1 with a central wavelength of, for example, 275 nanometers; or, the light source 230 is, for example, a halogen light source with a wide wavelength range which may emit the detection light L1, for example, visible light-near infrared light (VIS-NIR). The light sensor herein is, for example, a gallium nitride (Gang) sensor. In addition, the embodiments of the disclosure do not limit the types of light sources and/or light sensors.

As illustrated in FIGS. 2B to 2C, the detection chamber device 210A further includes a first inlet 210a1, a second inlet 210a2, a third inlet 210a3, an exhaust port 210b and an opening 210d. The first inlet 210a1, the second inlet 210a2, the third inlet 210a3, the exhaust port 210b and the opening 210d communicate with the detection chamber 210c. The air (if any) within the detection chamber 210c may be discharged through the exhaust port 210b. The liquid within the detection chamber 210c may be discharged to an outside of the detection chamber device 210A through the opening 210d and/or an external gas may enter the detection chamber 210 through the opening 210d.

As illustrated in FIG. 2B, the pump module 220 is configured to transport the to-be-tested liquid W1, the first reagent W2 and the diluent W3 to the detection chamber 210c. The pump module 220 includes a first pump 221, a second pump 222 and a third pump 223. The first pump 221 is connected to the first inlet 210a1 and is configured to transport the to-be-tested liquid W1 to the detection chamber 210c from the to-be-tested liquid container11 through the first inlet 210a1. The second pump 222 is connected to the second inlet 210a2 and is configured to transport the first reagent W2 to the detection chamber 210c from the first reagent container 12 through the second inlet 210a2. The third pump 223 is connected to the third inlet 210a3 and is configured to transport the diluent W3 to the detection chamber 210c from the diluent container 13. The to-be-detected liquid W1 could be a river water, a reservoir water, a tower water, a wastewater, a sewage, a ditch water, discharge water r, a fish pond water, etc.

As illustrated in FIG. 2B, in an embodiment, the controller 250 may control the volume of the to-be-tested liquid W1 entering the detection chamber 210c by controlling the time which the first pump 221 transports the to-be-tested liquid W1. For example, if a flow rate of the first pump 221 for the to-be-tested liquid W1 is q1 milliliter per second (ml/s), a transporting time of the first pump 221 for the to-be-tested liquid W1 may last m1/q1 seconds, wherein m1 represents the volume of the to-be-tested liquid W1 transported m1. The controller 250 may control the volume of the first reagent W2 entering the detection chamber 210c by controlling the time which the second pump 222 transport the first reagent W2. For example, if a transporting flow rate of the second pump 222 to the first reagent W2 is q2 ml/s, the transporting time of the second pump 222 for the first reagent W2 may last m2/q2 seconds, wherein m2 represents a first reagent volume m2 of the first reagent W2.

As illustrated in FIG. 2B, the motor 260 is, for example, a pneumatic motor. The motor 260 may be connected to the opening 210d of the detection chamber device 210A through the evacuation valve 270 to transport the air A1 into the detection chamber 210c through the opening 210d. The air A1 may be applied to the liquid within the detection chamber 210c to evenly mix the liquid within the detection chamber 210c.

As illustrated in FIG. 2B, the evacuation valve 270 is, for example, a three-way evacuation valve which includes a valve inlet 270a1, a valve opening 270a2 and a valve outlet 270a3. The motor 260 is connected to the valve inlet 270a1, and the air A1 enters the detection chamber 210c through the valve inlet 270a1, the valve opening 270a2 and the opening 210d of the detection chamber device 210A. When one of the valve inlet 270a1 and the valve outlet 270a3 is open, the other one of the valve inlet 270a1 and the valve outlet 270a3 is closed. For example, when the valve inlet 270a1 is opened, the valve outlet 270a3 is closed, and the air A1 provided by the motor 260 enters the evacuation valve 270 through the valve inlet 270a1, but does not leak through the valve outlet 270a3. When the valve outlet 270a3 is opened, the valve inlet 270a1 is closed, and the liquid within the detection chamber 210c is discharged through the opening 210d of the detection chamber device 210A, and the valve opening 20a2 and the valve outlet 270a3, but does not leak to the motor 260 through the valve inlet 270a1. In an embodiment, the air A1 is, for example, compressed air.

As illustrated in FIG. 2B, in an embodiment, the controller 250 opens the valve inlet 270a1 of the evacuation valve 270 and closes the valve outlet 270a3, and then controls the motor 260 to supply the air A1 to the valve inlet 270a1. The compressed air A1 enters the detection chamber 210c through the valve inlet 270a1, the valve opening 270a2 and the opening 210d of the detection chamber device 210A to evenly mix the to-be-tested liquid W1 and the first reagent W2 within the detection chamber 210c.

As illustrated in FIGS. 2A, 2B and 2D, the controller 250 is configured to perform the water-quality detection method, which includes the following procedures: a basic-voltage acquisition procedure S210, an initialization procedure S220, a voltage sensing procedure S230 and a dilution determination procedure S240. In the voltage sensing procedure S230, the controller 250 reads the detection voltage value VS of the detection voltage generated the light sensor 240. In the basic-voltage acquisition procedure S210, the controller 250 obtains the basic-voltage, and the operation will be described in detail later.

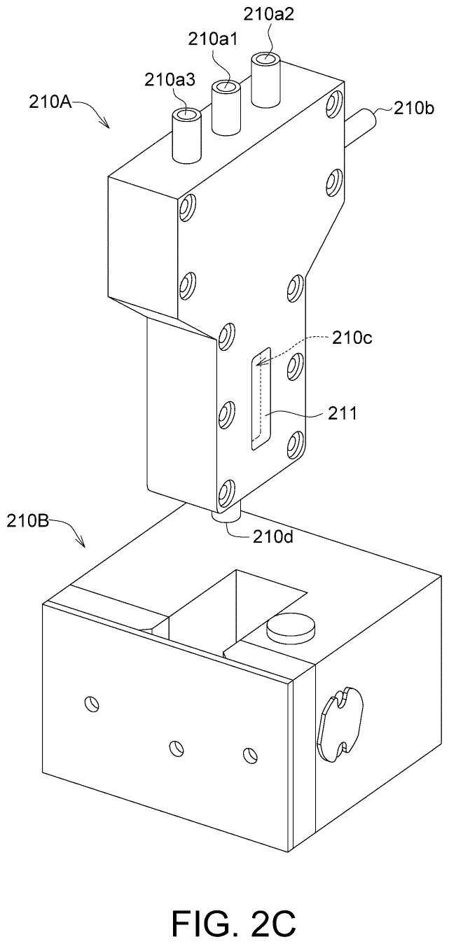

Referring further to FIG. 4, FIG. 4 illustrates a flow chart of the initialization procedure S220 in FIG. 2D. The initialization procedure S220 includes step S221 and step S222. In step S221, the controller 250 sets the dilution rate to 1. In step S222, the controller 250 drives the pump module 220 to transport the to-be-tested liquid W1 and the first reagent W2 into the detection chamber 210c.

In the basic-voltage acquisition procedure S210, the controller 250 obtains the basic-voltage value Vo. In the dilution determination procedure S240, the controller 250 calculates the voltage ratio VR of the detection voltage value VS to the basic-voltage value Vo and compares the voltage ratio VR with the critical value. When the voltage ratio VR is greater than the critical value, the concentration acquisition procedure S250 is performed; when the voltage ratio VR is not greater than the critical value, the to-be-tested liquid dilution procedure S260 is performed.

In the to-be-tested liquid dilution procedure S260, the controller 250 updates the dilution rate according to the voltage ratio VR, and drives the pump module 220 to transport the diluent W3, the to-be-tested liquid W1 and the first reagent W2, so that the volume sum of the to-be-tested liquid volume of the to-be-tested liquid W1 within the detection chamber 210c and the diluent volume of the diluent W3 to the to-be-tested liquid volume of the to-be-tested liquid W1 is equal to the dilution rate, and the voltage sensing procedure S230 and the dilution determination procedure S240 are performed again.

In the concentration acquisition procedure S250, the detection concentration value S1 of the to-be-tested liquid W1 is obtained according to the dilution rate and the detection voltage value. As a result, the concentration of the to-be-tested liquid W1 (for example, ammonia nitrogen concentration, nitrate nitrogen concentration) may be detected through optical means, and it is ensured that the liquid concentration in the detection chamber 210c is within the range that the light sensor 240 may accurately measure, and an applicable detection concentration range of the water-quality detection apparatus 200 may be effectively expanded.

Referring to FIGS. 3A and 3B, FIG. 3A illustrates a flow chart of the basic-voltage acquisition procedure S210 in FIG. 2D, and FIG. 3B illustrates a schematic diagram of the detection chamber 210c of the detection chamber device 210A covered with a light-shielding component 20.

The basic-voltage acquisition procedure S210 includes step S211 and step S212. In step S211, under the circumstances that the detection chamber 210c is covered with the light-shielding component 20 (for example, covering the entire first detection window 211), the controller 250 controls the light source 230 to emit the detection light L1. In the embodiment, the light-shielding component 20 is, for example, a black tape. In step S211, the detection chamber 210c may be evacuated, for example, without any liquid (the reagent, the to-be-tested liquid, the diluent, etc.). Due to the shielding by the light-shielding component 20, only a small amount of the detection light L1 is incident on the light sensor 240.

Next, step S212 is performed. In step S212, the controller 250 reads the detection voltage value VS of the detection voltage generated by the light sensor 240, and uses the detection voltage value VS obtained in this step as the basic-voltage value Vo. The basic-voltage value Vo may be stored in a memory (not illustrated), wherein the memory may be disposed within the controller 250, or may be disposed outside the controller 250 and electrically connected to the controller 250.

In an embodiment, the basic-voltage acquisition procedure S210 may be performed once when the water-quality detection apparatus 100 is first operated or after calibration. Subsequent concentration detection for the to-be-tested liquid W1 does not require to repeatedly perform the basic-voltage acquisition procedure S210.

The following is a further example of the initialization procedure S220 with FIGS. 2A, 2B, 2D and 4.

The initialization procedure S220 of the embodiment includes step S221 and step S222. In step S221, the controller 250 sets the dilution rate to 1, and in step S222, the first pump 221 and the second pump 222 transport the to-be-tested liquid W1 within the to-be-tested liquid container 11 and the first reagent W2 within the first reagent container 12 to the detection chamber 210c respectively.

For example, the detection chamber 210c has a detection chamber volume M, such as 10 ml. In step S222, the first pump 221 transports the to-be-tested liquid W1 with the to-be-tested liquid volume m1 to the detection chamber 210c. The to-be-tested liquid volume m1 is, for example, 10 ml. That is, in the initialization procedure S220 of the embodiment, the to-be-tested liquid volume m1 of the to-be-tested liquid W1 transported into the detection chamber 210c may be substantially equal to the detection chamber volume M of the detection chamber 210c (that is, the dilution rate is set to 1). The second pump 222 transports a first reagent W2 with a first reagent volume m2 into the detection chamber 210c. In step S222 of the embodiment, the first reagent volume m2 of the first reagent W2 transported to the detection chamber 210c and the to-be-tested liquid volume m1 satisfy the following formula (1). In formula (1), R2 is, for example, an integer greater than 1, for example, 10. Taking 10 ml of the to-be-tested liquid volume m1 and R2 being as 10 as an example, the first reagent volume m2 of the first reagent W2 transported to the detection chamber 210c is 1 ml.

$$\frac{m 1}{m 2} = R 2 \qquad ( 1 )$$

In another embodiment of the initialization procedure S220, in order to uniformly mix the to-be-tested liquid W1 and the first reagent W2 in the detection chamber 210c, after step S222 is completed, the controller 250 may further control the operation of the motor 260 to allow the air A1 to pass through the liquid within the detection chamber 210c is mixed evenly.

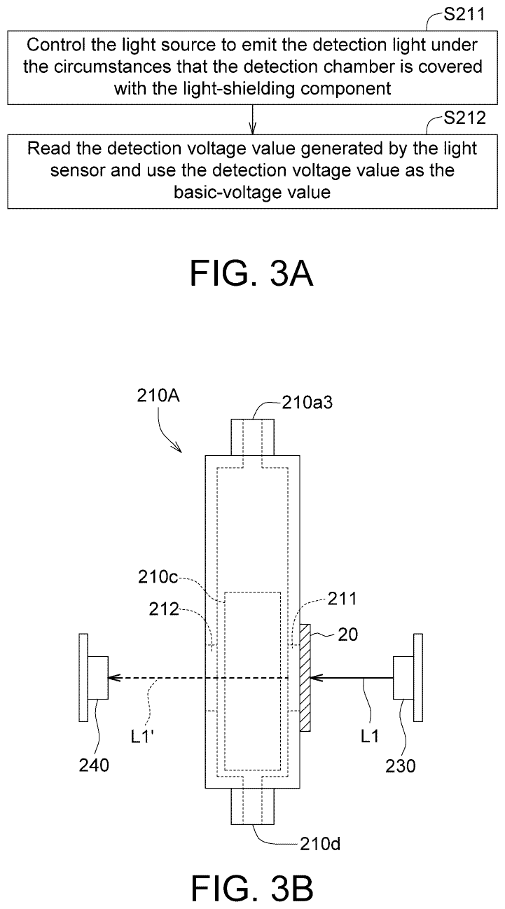

Referring to FIGS. 2A, 2B, 2D and 6, FIG. 6 illustrates a flow chart of the voltage sensing procedure S230 in FIG. 2D. The voltage sensing procedure S230 includes step S231, step S232 and step S233. In step S231, the controller 250 controls the light source 230 to emit the detection light L1. In step S232, the light sensor 240 senses the detection light L1′ traveling through the detection chamber 210c and generates the detection voltage value VS. In step S233, the controller 250 reads the detection voltage value VS from the light sensor 240.

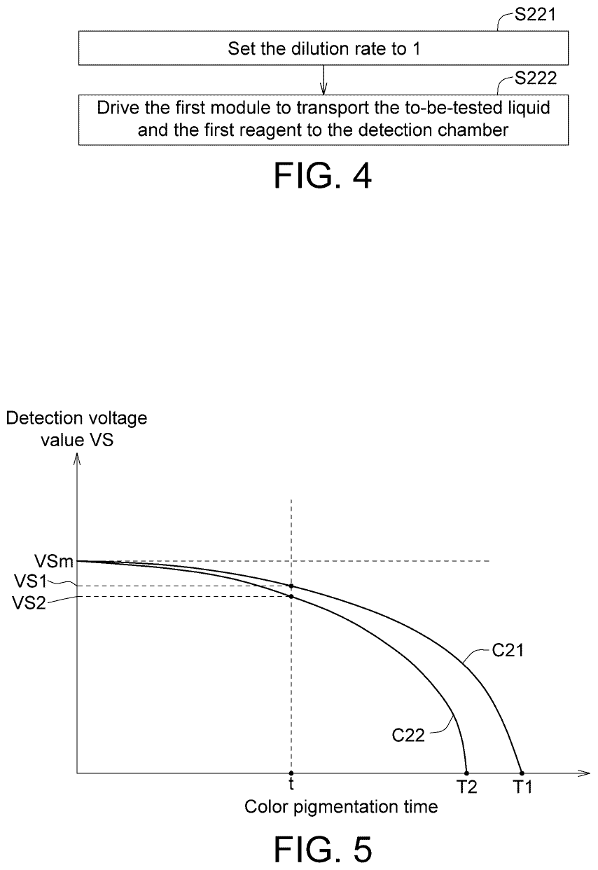

Referring to FIGS. 2A, 2B, 2D and 7, FIG. 7 illustrates a flow chart of the dilution determination procedure S240, the concentration acquisition procedure S250 and the to-be-tested liquid dilution procedure S260 in FIG. 2D. The dilution determination procedure S240 includes step S241 and step S242. In step S241, the controller 250 calculates the voltage ratio VR of the detection voltage value VS to the basic-voltage value Vo (i.e., VR=VS/Vo). In step S242, the controller 250 determines whether the voltage ratio VR is greater than a critical value. If yes, the concentration acquisition procedure S250 is performed; if not, the to-be-tested liquid dilution procedure S260 is performed.

In the to-be-tested liquid dilution procedure S260 of the embodiment, the controller 250 may drive the pump module 220 to transport the diluent W3 to dilute the to-be-tested liquid W1, so that the concentration of the to-be-tested liquid W1 within the detection chamber 210c is within the effective detecting range of the water-quality detection apparatus 100. Furthermore, the controller 250 determines and updates the dilution rate according to the voltage ratio VR, and in order to make the concentration of the to-be-tested liquid W1 within the detection chamber 210c equal to the dilution rate, the controller 250 drives the pump module 220 to transport the diluent W3 to the detection chamber 210c according to the dilution rate, or further transport the first reagent W2 according to the volume of the liquid within the detection chamber 210c.

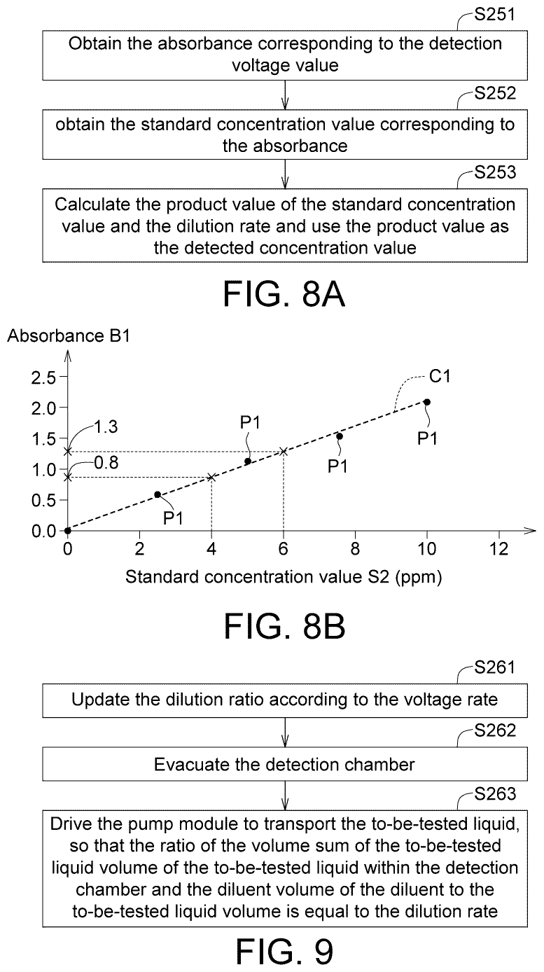

Referring to FIGS. 2A, 2B, 2D and 9, FIG. 9 illustrates a flow chart of the to-be-tested liquid dilution procedure S260 in FIG. 2D according to another embodiment. The to-be-tested liquid dilution procedure S260 in the embodiment includes step S261, step S262 and step S263. In step S261, the controller 250 obtains and updates the dilution rate R1 according to the voltage ratio VR. For example, based on a relationship between the voltage ratio and the dilution rate, the controller 250 obtains the corresponding dilution rate R1 according to the voltage ratio VR, and updates the dilution rate. The relationship between voltage ratio and dilution rate is, for example, a table, equation, etc. The relationship between the voltage ratio and the dilution rate may be stored in a memory (not illustrated), wherein the memory is, for example, disposed within the controller 250, or configured outside the controller 250 and electrically connected to the controller 250.

For example, Referring to Table 1 below, which lists the relationship between the voltage ratio VR and the dilution rate R1 according to an embodiment of the disclosure. The relationship between the value of the voltage ratio VR and the value of the dilution rate R1 depends on the type of the to-be-tested liquid W1 and is not limited in the embodiment of the disclosure. The dilution rate R1 is not greater than the maximum dilution rate. In a comparative example, when the dilution procedure is performed with the dilution rate R1 that is equal to or greater than the maximum dilution rate, an error of the obtained detection concentration value compared with the standard concentration value will exceed an allowable value (the error is too large), and thus it is preferable for the dilution rate R1 to being no larger than the maximum dilution rate. In addition, the maximum dilution rate depends on the capability or performance of the water-quality detection apparatus 200 and/or the type of the to-be-tested liquid W1, which is not limited in the embodiment of the disclosure.

TABLE 1

| voltage ratio VR | dilution rate R1 |
|---|---|
| 1 | 20 |
| 1.3 | 10 |
| 1.7 | 5 |
| 2 | 2 |

Before the pump module 220 transports the to-be-tested liquid W1, the first reagent W2, and the diluent W3, step S262 is performed to evacuate the detection chamber 210c. For example, in step S262, the controller 250 controls the valve outlet 270a3 of the evacuation valve 270 to open and close the valve inlet 270a1, so that the liquid within the detection chamber 210c, for example, the mixed liquid of the to-be-tested liquid W1 and the first reagent W2, may passes through the valve outlet 270a3 to be discharged out of the detection chamber 210c by its own weight. For example, the valve outlet 270a3 of the evacuation valve 270 is open for a period of time (for example, a few seconds), thereby ensuring that the liquid within the detection chamber 210c is completely discharged from the detection chamber 210c, the controller 250 closes the valve outlet 270a3 and opens the valve inlet 270a1.

In step S263, the controller 250 drives the pump module 220 to transport the to-be-tested liquid W1 in the to-be-tested liquid container11, the first reagent W2 in the first reagent container 12 and the diluent W3 in the container 13 to the detection chamber 210c according to the updated dilution rate R1 in step S261. A ratio of the volume sum of the to-be-tested liquid volume m1 of the to-be-tested liquid W1 within the detection chamber 210c and the diluent volume m3 to the to-be-tested liquid volume m1 is equal to the dilution rate R1, that is, the following formula (2) is satisfied.

$$\frac{( m 1 + m 3 )}{m 1} = R 1 \qquad ( 2 )$$

In the embodiment, the dilution rate R1 is equal to 2 as an example. The to-be-tested liquid volume m1 of the to-be-tested liquid W1 transported to the detection chamber 210c by the pump module 220 is 5 ml, and the diluent volume m3 of the diluent W3 transported to the detection chamber 210c is 5 ml, so that the ration of the volume sum (m1+m3 equals to 10 ml) of the to-be-tested liquid volume m1 of the to-be-tested liquid W1 in the detection chamber 210c and the diluent volume m3 of the diluent W3 in the detection chamber 210c to the to-be-tested liquid volume m1 (equal to 5 ml) of the to-be-tested liquid W1 is equal to 2. At this time, a liquid composition in the detection chamber 210c complies with the dilution rate R1 which is equal to 2.

The to-be-tested liquid volume m1 of the to-be-tested liquid W1 and the diluent volume m3 of the diluent W3 in the detection chamber 210c and the first reagent volume m2 of the first reagent W2 satisfy the following formula (3). R2 in the formula (3) is a reagent addition ratio, and the reagent addition ratio R2 is the value is a real number less than 1.

$$\frac{m 2}{( m 1 + m 3 )} = R 2 \qquad ( 3 )$$

For example, taking the reagent addition ratio R2 being 10% as an example, when the to-be-tested liquid volume m1 of the to-be-tested liquid W1 in the detection chamber 210c is 5 ml and the diluent volume m3 of the diluent W3 is 5 ml, the first reagent volume m2 of the first reagent W2 in the detection chamber 210c must be 1 ml to comply with the reagent addition ratio R2 is equal to 10%.

After performing the to-be-tested liquid dilution procedure S260, the controller 250 performs the voltage sensing procedure S230 again, that is, the process returns to the voltage sensing procedure S230 (as illustrated in FIG. 2D).

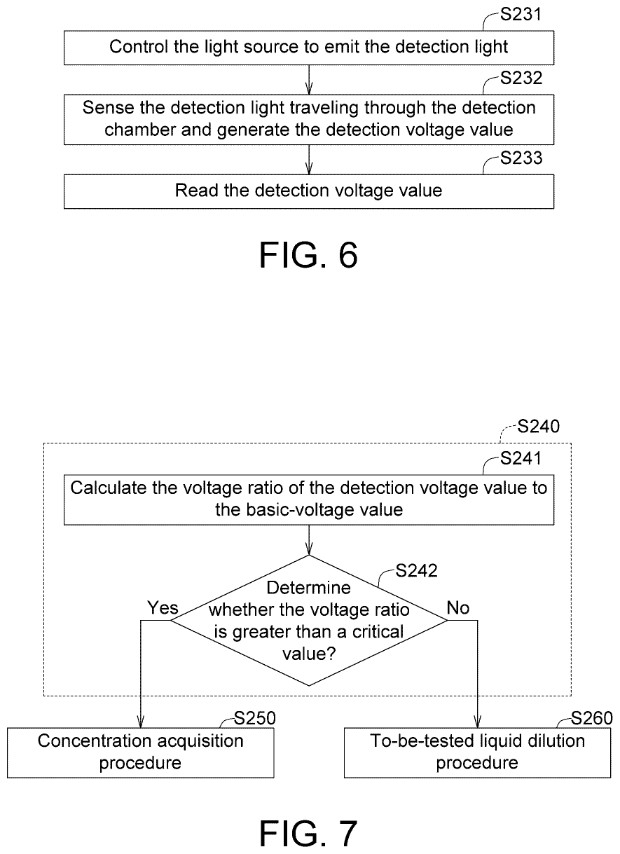

In the dilution determination procedure S240, when the controller 250 determines that the voltage ratio VR is greater than the critical value, the concentration acquisition procedure S250 is performed. The concentration acquisition procedure S250 will be described in detail below. Referring to FIGS. 2A, 2B, 8A and 8B, FIG. 8A illustrates a flow chart of the concentration acquisition procedure S250 in FIG. 2D, and FIG. 8B illustrates a relationship diagram between a standard concentration value S2 of the to-be-tested liquid W1 and an absorbance B1 according to an embodiment of the disclosure (calibration curve).

In the concentration acquisition procedure S250, the controller 250 obtains the detection concentration value S1 of the to-be-tested liquid W1 according to the dilution rate and the detection voltage value VS. The concentration acquisition procedure S250 includes steps S251, S252 and S253, and they will be further described below with examples.

In step S251, the controller 150 obtains the corresponding absorbance B1 according to the detection voltage value VS by the Beer-Lambert law.

In step S252, the controller 250 obtains the standard concentration value S2 corresponding to the absorbance B1 according to the absorbance B1. For example, if the absorbance B1 is 1.3, the corresponding standard concentration value S2 is 6 based on the relationship illustrated in FIG. 8B.

In addition, a relationship between the standard concentration value S2 and the absorbance B1 in FIG. 8B may be represented by the curve C1. The equation of the curve C1 is, for example, represented by B1=a×S2+b, wherein a and b are constants, which are not limited by the embodiment of the disclosure. In an embodiment, the curve C1 is, for example, a linear fitting equation of multiple data points P1, wherein the data points P1 are obtained, for example, through experiments or simulations. The relationship between the standard concentration value S2 and the absorbance B1 may be obtained in advance and stored in a memory (not illustrated), wherein the memory is, for example, disposed within the controller 250, or is disposed outside the controller 150 and is electrically connected to the controller 250.

In step S253, the controller 250 calculates a product value of the standard concentration value S2 obtained in step S252 and the dilution rate R1 and uses the product value as the detected concentration value S1, as illustrated in the following formula (4). Furthermore, due to the to-be-tested liquid W1 being diluted by the diluent W3, the actual detection concentration value S1 of the to-be-tested liquid W1 is equal to the product of the standard concentration value S2 and the dilution rate R1.

$$S 1 = S 2 \times R 1 \qquad ( 4 )$$

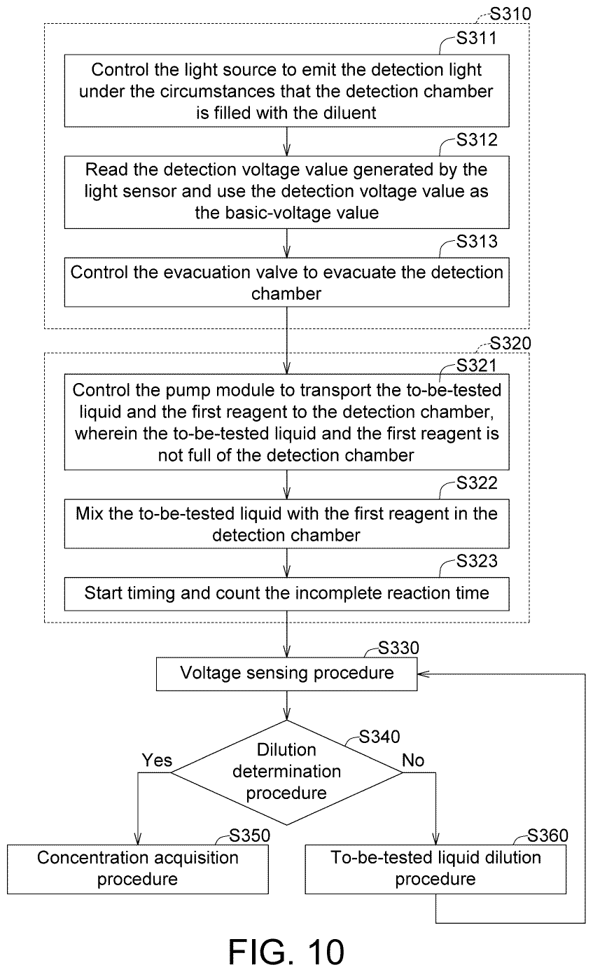

Referring to FIG. 10, FIG. 10 illustrates a schematic diagram of the water-quality detection method of the water-quality detection apparatus 200 in FIG. 2B according to another embodiment. The water-quality detection method of the embodiment includes the steps the same as or similar to that of the aforementioned water-quality detection method in FIG. 2D. The differences are described below.

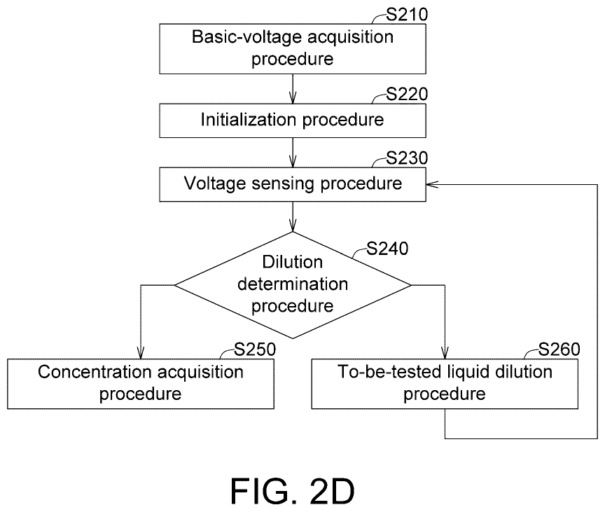

Referring to FIG. 5, FIG. 5 illustrates a relationship diagram between a color pigmentation time of the to-be-tested liquid W1 and the detection voltage value VS according to the embodiment of the disclosure. The curve C21 represents a relationship between the color pigmentation time of the to-be-tested liquid W1 with a lower concentration and the detection voltage value VS, and the curve C22 represents a relationship between the color pigmentation time and the detection voltage value VS of the to-be-tested liquid W1 with a higher concentration. It may be seen from the figure that in the early stage of mixing the reagent with the to-be-tested liquid W1, the reagent and the to-be-tested liquid W1 have not yet reacted, so the detection voltage value VS is the highest (i.e., the maximum detection voltage value Vamp). As time progresses, the reagent and the to-be-tested liquid W1 gradually react until complete reaction. The complete reaction time of the curve C21 (lower concentration to-be-tested liquid W1) is T1, and the complete reaction time of the curve C22 (higher concentration to-be-tested liquid W1) is T2. Comparing the curves C21 and C22, it may be seen that the higher the concentration of the to-be-tested liquid W1 is, the shorter the complete reaction time is. In addition, the color (color change) of the to-be-tested liquid W1 changes depending on its impurity concentration. For example, the higher the impurity concentration is, the darker the color after complete reaction is.

Referring to FIGS. 2A, 2B and 10, the basic-voltage acquisition procedure S310 of the embodiment includes step S311, step S312 and step S313, and they will be further described below with examples.

In step S311, under the circumstances that the detection chamber 210c is filled with the diluent W3, the light source 230 is controlled to emit the detection light L1. In step S312, the controller 250 reads the detection voltage value VS generated by the light sensor 240, and uses the detection voltage value VS as the basic-voltage value Vo. The light sensor 240 receives the detection light L1′ traveling through the detection chamber 210c and generates the detection voltage, and the controller 250 further reads the detection voltage value VS from the light sensor 240 and sets the basic-voltage value Vo as the detection voltage value VS. The basic-voltage value Vo is, for example, substantially equal to the detection voltage value vamp in FIG. 5. This basic-voltage value Vo may also be called a diluent voltage value. In step S313, the controller 250 controls the evacuation valve 270 to evacuate the detection chamber 210c.

The initialization procedure S320 in the embodiment includes step S321, step S322 and step S323. In step S321, the controller 250 controls the pump module 220 to transport the to-be-tested liquid W1 in the to-be-tested liquid container 11 and the first reagent W2 in the first reagent container 12 to the detection chamber 210c, wherein the to-be-tested liquid W1 and the first reagent W2 is not full of the detection chamber 210c.

For example, the first pump 221 transports the to-be-tested liquid W1 in the to-be-tested liquid container 11 to the detection chamber 210c. In an embodiment, the detection chamber volume M of the detection chamber 210c and the to-be-tested liquid volume m1 of the to-be-tested liquid W1 transported to the detection chamber 210c satisfy the following formula (5). In the formula (5), R1ₘₐₓ represents the maximum dilution rate. It may be seen from equation (5) that since the to-be-tested liquid W1 transported to the detection chamber 210c does not fill the detection chamber 210c, there is still a dilutable space in the detection chamber 210c.

$$m 1 \leq \frac{M}{R {1}_{max}} \qquad ( 5 )$$

Taking the detection chamber volume M of the detection chamber 210c being 10 ml and the maximum dilution rate R1ₘₐₓ being 5 as an example, the to-be-tested liquid volume m1 of the to-be-tested liquid W1 transported to the detection chamber 210c may be 2 ml.

The second pump 222 transports the first reagent W2 in the first reagent container 12 to the detection chamber 210c. The first reagent volume m2 of the first reagent W2 and the to-be-tested liquid volume m1 of the to-be-tested liquid W1 satisfy the following formula (6). Taking the the to-be-tested liquid volume m1 being 2 ml and the reagent addition ratio R2 being 10% as an example, in order to comply with the reagent addition ratio R2 being 10%, the first reagent volume m2 transported to the detection chamber 210c must be 0.2 ml.

$$R 2 = \frac{m 2}{m 1} \qquad ( 6 )$$

In step S322, the to-be-tested liquid W1 and the first reagent W2 in the detection chamber 210c are mixed. The mixing method has been described above and it will not be repeated again here.

In step S323, the controller 250 counts an incomplete reaction time t. Furthermore, in step S323, the controller 250 starts a timer, and proceeds to the next procedure after the incomplete reaction time t has elapsed. The incomplete reaction time t is less than a complete reaction time T1 of the to-be-tested liquid W1 and the first reagent W2. After step S323, the controller 250 performs the voltage sensing procedure S330, and the voltage sensing procedure S330 of the embodiment includes the steps the same as that of the previous embodiment, and it will not be described again. In short, after waiting for the incomplete reaction time t (the incomplete reaction time t is illustrated in FIG. 5), the controller 250 controls the light source 230 to emit the detection light L1 and reads the detection voltage value VS from the light sensor 140. As illustrated in FIG. 8B, the incomplete reaction time t is less than the complete reaction time T1. Therefore, in the embodiment, there is no need to wait for the complete reaction of the to-be-tested liquid W1 and the first reagent W2, and thus it may save a lot of detection time. In an embodiment, the incomplete reaction time t is, for example, the color pigmentation time when the mixed liquid may be detected.

After obtaining the detection voltage value VS, the controller 250 performs the dilution determination procedure S340. The dilution determination procedure S340 of the embodiment includes the steps similar to that of the aforementioned dilution determination procedure S250, and whether the voltage ratio VR is greater than the critical value is determined. If so, the concentration acquisition procedure S350 is performed. If not, the to-be-tested liquid dilution procedure S360 is performed.

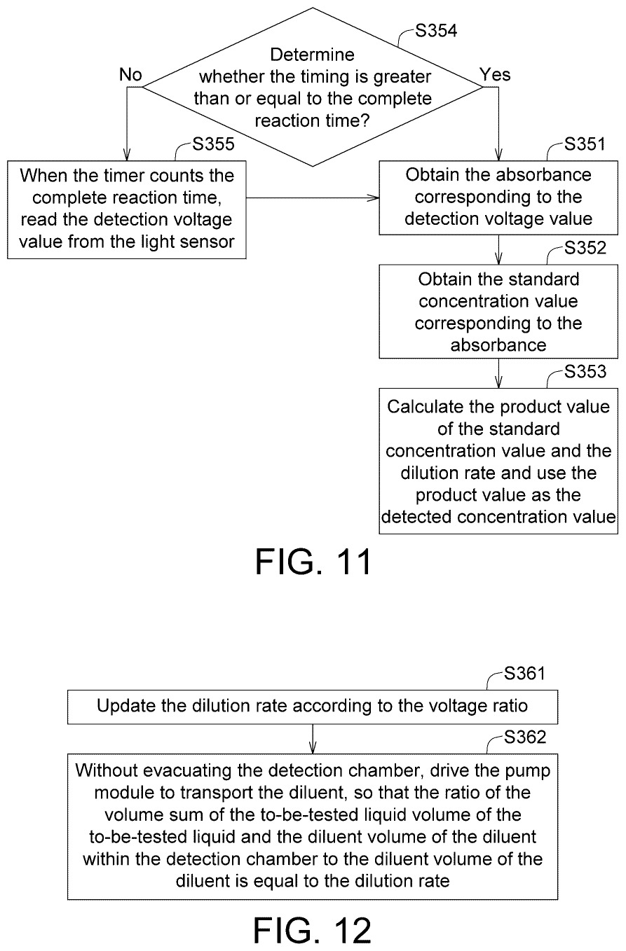

Referring to FIGS. 2A, 10 and 11, FIG. 11 illustrates a flow chart of the concentration acquisition procedure S350 in FIG. 10. The concentration acquisition procedure S350 in the embodiment includes step S354, step S355, step S351, step S352 and step S353. In step S354, the controller 250 determines whether the timer counts time which is greater than or equal to the complete reaction time T1. If so, step S351, step S352 and step S353 are performed in sequence. If the timer counts time which is less than the complete reaction time T1, step S355 is performed. In step S355, when the timer counts the complete reaction time T1, the controller 250 reads the detection voltage value VS from the light sensor 240 to obtain the detection voltage value VS after the first reagent W2 is completely reacted. After step S354 is completed, the controller 250 performs step S351, step S352 and step S353 in sequence. Steps S351, S352, and S353 of the embodiment are the same as or similar to steps S251, S252, and S253 of the aforementioned concentration acquisition procedure S250 respectively, and they will not be repeated again here.

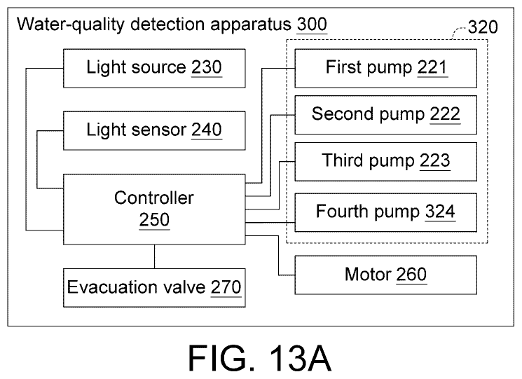

Referring to FIGS. 2A, 2B, 10 and 12, FIG. 12 illustrates a flow chart of the to-be-tested liquid dilution procedure S360 in FIG. 10. The to-be-tested liquid dilution procedure S360 in the embodiment includes step S361 and step S362. In step S361, the controller 250 updates the dilution rate R1 according to the voltage ratio VR. For example, the controller 250 obtains the dilution rate R1 corresponding to the voltage ratio VR according to the relationship between the voltage ratio and the dilution rate. The relationship between the voltage ratio and the dilution rate is, for example, a table, equation, etc. The relationship between the voltage ratio and the dilution rate may be stored in a memory (not shown), wherein the memory is, for example, disposed in the controller 250, or disposed outside the controller 250 and electrically connected to the controller 250. In the embodiment, the dilution rate R1 obtained in the dilution procedure being 2 in Table 1-2 is taken as an example.

In step S362, without evacuating the detection chamber 210c, the controller 250 drives the pump module 200 to transport the diluent W3, so that the ratio of the volume sum of the to-be-tested liquid volume of the to-be-tested liquid W1 and the diluent volume of the diluent W3 within the detection chamber 210c to the diluent volume of the diluent W3 is equal to the dilution rate R1.

For example, the first pump 221 transports the diluent W3 in the diluent container 13 to the detection chamber 210c to dilute the to-be-tested liquid W1 in the detection chamber 210c. The dilution rate R1, the diluent W3 transported to the detection chamber 210c, and the to-be-tested liquid W1 in the detection chamber 210c satisfy the above formula (2).

Taking the to-be-tested liquid volume m1 of the to-be-tested liquid W1 in the detection chamber 210c being 2 ml and the dilution rate R1 being 2 as an example, in order to comply with the dilution rate R1 is equal to 2, the diluent volume m3 of the diluent W3 transported to the detection chamber 210c is 2 ml. After the first pump 221 transports the diluent W3 in the diluent container 13 to the detection chamber 210c, there are 2 ml of the to-be-tested liquid W1, 2 mL of the diluent W3 and 0.2 ml of the first reagent W2 in the detection chamber 210c, and the diluted mixture is 4.2 ml.

In addition, the second pump 222 transports the first reagent W2 in the first reagent container 12 to the detection chamber 210c. The first reagent volume Δm2 of the first reagent W2 transported to the detection chamber 210c, the to-be-tested liquid volume m1 of the to-be-tested liquid W1 in the detection chamber 210c, the diluent volume m3 of the diluent W3 in the detection chamber 210c, and the reagent addition ratio R2 satisfies the following formula (7).

$$Δ m 2 = ( ( m 1 + m 3 ) \times R 2 ) - m 2 ) \qquad ( 7 )$$

Taking the to-be-tested liquid volume m1 of the to-be-tested liquid W1 in the detection chamber 210c being 2 ml, the diluent volume m3 of the diluent W3 in the detection chamber 210c being 2 ml and the reagent addition ratio R2 is 10% as an example, the first reagent volume Δm2 of the first reagent W2 is required to be transported to the detection chamber 210c is 0.2 ml to make the reagent addition ratio R2 be equal to 10%. After the first pump 221 transports the first reagent W2 to the detection chamber 210c, the to-be-tested liquid W1 in the detection chamber 210c is 2 ml, the diluent W3 in the detection chamber 210c is 2 ml, and the first reagent W2 in the detection chamber 210c is 0.4 ml, wherein the diluted mixture is 4.4 ml.

Then, the aforementioned method may be used to mix the to-be-tested liquid W1, the first reagent W2 and the diluting liquid W3 in the detection chamber 210c into the diluted mixture. Then, the process returns to the voltage sensing procedure S330.

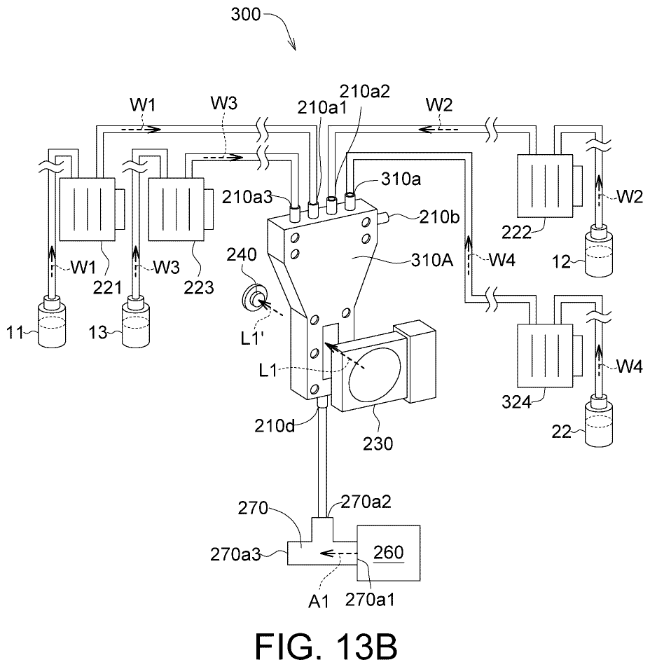

Referring to FIGS. 13A and 13B, FIG. 13A illustrates a functional block diagram of a water-quality detection apparatus 300 according to another embodiment of the disclosure, and FIG. 13B illustrates a schematic diagram of the water-quality detection apparatus 300 in FIG. 13A.

As illustrated in FIGS. 13A and 13B, the water-quality detection apparatus 300 includes a detection chamber device 310A, a pump module 320, the light source 230, the light sensor 240, the controller 250, the motor 260 and the evacuation valve 270. The light source 230 and the light sensor 240 may be disposed in the detection base (not illustrated). For example, the detection window may be disposed in a recess of the detection base to prevent the detection from being interfered by ambient light.

The water-quality detection apparatus 300 includes the technical features the same as or similar to that of the aforementioned water-quality detection apparatus 100, and at least one difference is that the detection chamber device 310A of the water-quality detection apparatus 300 further has a fourth inlet 310a, and the pump module 320 further includes a fourth pump 324. The fourth pump 324 is connected to the fourth inlet 310a and is configured to transport the second reagent W4 in the second reagent container 22 into the inside of the detection chamber device 310A through the fourth inlet 310a. The first reagent W2 and the second reagent W4 may respectively display colors for different substances in the to-be-tested liquid W1. In other embodiment, the first reagent W2 and the second reagent W4 may be two reagents used to detect a specific single substance in the to-be-tested liquid W1. For example, in the case of detecting ammonia nitrogen with API reagents, the first reagent W2 is a coloring reagent, and the second reagent W4 is a reaction reagent.

The water-quality detection method of the water-quality detection apparatus 300 includes the steps the same as or similar to that of the water-quality detection method of the water-quality detection apparatus 200, and at least one difference is that the transport step of the first reagent W2 may be added before or after the transport step of the second reagent W4.

In summary, the water-quality detection apparatus of the embodiment of the disclosure may detect the standard concentration value to be detected after diluting the to-be-tested liquid, and then obtain (or calculate) the detection concentration value of the to-be-tested liquid according to the dilution rate and the standard concentration value. As a result, the detection concentration range of water-quality detection apparatus may be expanded.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A water-quality detecting equipment, comprising:
a detection chamber device having a detection chamber;
a pump module configured to transport a to-be-tested liquid and a reagent to the detection chamber;
a light source configured to emit a detection light to the detection chamber;
a light sensor configured to sense the detection light traveling through the detection chamber and generate a detection voltage; and
a controller electrically connected to the light sensor and the pump module, and configured to perform following procedures:
a voltage sensing procedure, comprising reading a detection voltage value of the detection voltage from the light sensor;
a dilution determination procedure, comprising: calculating a voltage ratio of the detection voltage value to a basic-voltage value, comparing the voltage ratio with a critical value, performing a concentration acquisition procedure when the voltage ratio is greater than the critical value, and performing a to-be-tested liquid dilution procedure when the voltage ratio is not greater than the critical value;
wherein in the concentration acquisition procedure, a detection concentration value of the to-be-tested liquid is obtained according to a dilution rate and the detection voltage value;
wherein In the to-be-tested liquid dilution procedure, the dilution rate is updated according to the voltage ratio, and the pump module is driven to transport a diluent by the controller, so that a ratio of a volume sum of a to-be-tested liquid volume of the to-be-tested liquid in the detection chamber and a diluent volume of the diluent to the to-be-tested liquid volume of the to-be-tested liquid is equal to the dilution rate, and the voltage sensing procedure and the dilution determination procedure are performed.

2. The water-quality detecting equipment according to claim 1, wherein the controller is further configured to perform an initialization procedure, comprising:
setting the dilution rate to 1; and
driving the pump module to transport the to-be-tested liquid and the reagent to the detection chamber.

3. The water-quality detecting equipment according to claim 1, wherein in the to-be-tested liquid dilution procedure, the controller is further configured to drive the pump module to transport the to-be-tested liquid and the reagent to the detection chamber according to the dilution rate.

4. The water-quality detecting equipment according to claim 3, further comprising an evacuation valve electrically connected to the controller, wherein in the to-be-tested liquid dilution procedure, before the pump module transports the diluent, the to-be-tested liquid and the reagent, the controller further controls the evacuation valve to evacuate the detection chamber.

5. The water-quality detecting equipment according to claim 3, wherein in the concentration acquisition procedure, the controller is further configured to:
obtain an absorbance corresponding to the detection voltage value;
obtain a standard concentration value corresponding to the absorbance; and
calculate a product of the standard concentration value and the dilution rate and use the product as the detection concentration value.

6. The water-quality detecting equipment according to claim 1, wherein the controller is further configured to perform a basic-voltage acquisition procedure, comprising:
controlling the light source to emit the detection light under the circumstances that the detection chamber is covered with a light-shielding component; and
reading the detection voltage value generated by the light sensor and using the detection voltage value as the basic-voltage value.

7. The water-quality detecting equipment according to claim 2, wherein the initialization procedure further comprises:
the controller starting timing, and performing the voltage sensing procedure after counting an incomplete reaction time, wherein the incomplete reaction time is less than a complete reaction time of the to-be-tested liquid and the reagent.

8. The water-quality detecting equipment according to claim 7, wherein the concentration acquisition procedure further comp rises:
before obtaining the detection concentration value of the to-be-tested liquid, determining whether the timing is greater than or equal to the complete reaction time, and if the timing is not greater than or not equal to the complete reaction time, again reading the detection voltage value generated by the light sensor; if the timing is greater than or equal to the complete reaction time, obtaining the detection concentration value of the to-be-tested liquid according to the detection voltage value.

9. The water-quality detecting equipment according to claim 8, further comprising an evacuation valve electrically connected to the controller, wherein the controller is further configured to perform a basic-voltage acquisition procedure, comprising:
controlling the light source to emit the detection light by the controller under the circumstances that the detection chamber is filled with the diluent;
reading the detection voltage value generated by the light sensor and using the detection voltage value as the basic-voltage value by the controller; and
controlling the evacuation valve to evacuate the detection chamber by the controller.

10. The water-quality detecting equipment according to claim 7, further comprising:
a to-be-tested liquid container configured to store the to-be-tested liquid; and
a reagent container configured to store the reagent; and
a diluent container configured to store the diluent;
wherein the controller is further configured to drive the pump module to transport the to-be-tested liquid to the detection chamber from the to-be-tested liquid container, and the controller is further configured to drive the pump module to transport the reagent to the detection chamber from the reagent container, and the controller is further configured to drive the pump module to transport the diluent to the detection chamber from the diluent container.

11. A water-quality detecting method, comprising:
transporting a to-be-tested liquid and a reagent to a detection chamber of a detection chamber device by a pump module;
emitting a detection light to the detection chamber by a light source;
sensing the detection light traveling through the detection chamber and generating a detection voltage by a light sensor;
performing a voltage sensing procedure by a controller, comprising: reading a detection voltage value of the detection voltage; and
performing a dilution determination procedure by the controller, comprising:
calculating a voltage ratio of the detection voltage value to a basic-voltage value;
comparing the voltage ratio with a critical value;
performing a concentration acquisition procedure when the voltage ratio is greater than the critical value; and
performing a to-be-tested liquid dilution procedure when the voltage ratio is not greater than the critical value;
wherein the concentration acquisition procedures comprises:
obtaining a detection concentration value of the to-be-tested liquid according to a dilution rate and the detection voltage value by the controller;
wherein the to-be-tested liquid dilution procedure comprises:
updating the dilution rate according to the voltage ratio by the controller; and
driving the pump module to transport a diluent by the controller, so that a ratio of a volume sum of a to-be-tested liquid volume of the to-be-tested liquid in the detection chamber and a diluent volume of the diluent to the to-be-tested liquid volume of the to-be-tested liquid is equal to the dilution rate; and
performing the voltage sensing procedure and the dilution determination procedure by the controller.

12. The water-quality detecting method according to claim 11, further comprising:
performing an initialization procedure by the controller, comprising:
setting the dilution rate to 1; and
driving the pump module to transport the to-be-tested liquid and the reagent to the detection chamber.

13. The water-quality detecting method according to claim 11, wherein the to-be-tested liquid dilution procedure comprises:
driving the pump module to transport the to-be-tested liquid and the reagent to the detection chamber according to the dilution rate by the controller.

14. The water-quality detecting method according to claim 13, wherein the to-be-tested liquid dilution procedure comprises:
before the pump module transports the diluent, the to-be-tested liquid and the reagent, controlling an evacuation valve to evacuate the detection chamber by the controller, wherein the evacuation valve is electrically connected to the controller.

15. The water-quality detecting method according to claim 13, wherein the concentration acquisition procedure comprises:
obtaining an absorbance corresponding to the detection voltage value by the controller;
obtaining a standard concentration value corresponding to the absorbance by the controller; and
calculating a product of the standard concentration value and the dilution rate and using the product as the detection concentration value by the controller.

16. The water-quality detecting method according to claim 11, further comprising:
performing a basic-voltage acquisition procedure by the controller, comprising:
controlling the light source to emit the detection light under the circumstances that the detection chamber is covered with a light-shielding component; and
reading the detection voltage value generated by the light sensor and using the detection voltage value as the basic-voltage value.

17. The water-quality detecting method according to claim 12, wherein the initialization procedure further comprises:
reading the detection voltage value after timing reaching an incomplete reaction time by the controller, wherein the incomplete reaction time is less than a complete reaction time of the to-be-tested liquid and the reagent.

18. The water-quality detecting method according to claim 17, wherein the concentration acquisition procedure comprises:
before obtaining the detection concentration value of the to-be-tested liquid, determining whether the timing is greater than or equal to the complete reaction time by the controller;
if the timing is not greater than or not equal to the complete reaction time, reading the detection voltage value generated by the light sensor again; and
if the timing is greater than or equal to the complete reaction time, obtaining the detection concentration value of the to-be-tested liquid according to the detection voltage value.

19. The water-quality detecting method according to claim 18, further comprising:
performing a basic-voltage acquisition procedure, comprising:
controlling the light source to emit the detection light by the controller under the circumstances that the detection chamber is filled with the diluent;
reading the detection voltage value generated by the light sensor and using the detection voltage value as the basic-voltage value by the controller; and
controlling an evacuation valve to evacuate the detection chamber by the controller, wherein the evacuation valve is electrically connected to the controller.

20. The water-quality detecting method according to claim 17, wherein a to-be-tested liquid container is configured to store the to-be-tested liquid, a reagent container is configured to store the reagent, and a diluent container is configured to store the diluent; the water-quality detecting method further comprises:
driving the pump module to transport the to-be-tested liquid to the detection chamber from the to-be-tested liquid container by the controller;
driving the pump module to transport the reagent to the detection chamber from the reagent container by the controller; and
driving the pump module to transport the diluent to the detection chamber from the diluent container by the controller.
